(19) United States
(12) Patent Application Publication
(10) Pub. No.: US 2020/0144480 A1
(43) Pub. Date: May 7, 2020

(54) Implantable Devices Based on Magnetoelectric Antenna, Energy Harvesting and Communication

(71) Applicant: Northeastern University, Boston, MA (US)

(72) Inventors: Nian Xiang Sun, Winchester, MA (US); Mohsen Zaeimbashi, Somerville, MA (US); Hwaider Lin, Quincy, MA (US)

(21) Appl. No.: 16/672,167

(22) Filed: Nov. 1, 2019

Related U.S. Application Data
(60) Provisional application No. 62/754,145, filed on Nov. 1, 2018.

(51) Int. Cl. H01L 41/12; H01Q 1/22; A61N 1/378; H01Q 1/24; A61N 1/372

(52) U.S. Cl. CPC H01L 41/12; H01Q 1/2283; A61N 1/37229; H01Q 1/248; A61N 1/3787

(57) ABSTRACT

Disclosed is an implantable system that comprises a magnetoelectric (ME) antenna, a radio frequency rectifier, and a transmitter. The ME antenna may be characterized by a resonance frequency that changes according to an ambient magnetic field strength. The radio frequency rectifier may be configured to convert radio frequency energy, received by the ME antenna, into a direct current voltage, and to direct the direct current voltage to a storage capacitor. The transmitter may be configured to apply a transmission signal to the ME antenna. A transceiver may communicate with one or more of the implantable systems, to provide radio frequency energy to the implantable devices for energy harvesting, and to receive transmitted information from the implantable systems. The implantable system may be disposed within a brain to detect neuronal activity, by detecting small magnetic fields generated by such neuronal activity.

### RELATED APPLICATION(S)

This application claims the benefit of U.S. Provisional Application No. 62/754,145, filed on Nov. 1, 2018. The entire teachings of the above application are incorporated herein by reference.

### GOVERNMENT SUPPORT

This invention was made with government support under Grant No. 1160504 from the National Science Foundation and Grant No. D15PC00009 from DARPA. The government has certain rights in the invention.

### BACKGROUND

Important aspects of human brain research are the ability to record brain signals, and to stimulate neurons, with a very high spatial and temporal resolution. To date, many tethered and tether-less devices have been developed for neural modulation purposes. It can be a great challenge to obtain a high density distributed network of tethered (optical or electrical) devices due to wiring and interconnection constraints related to such devices. Tethered devices may also suffer from safety problems related to bio-compatibility and insulation of high density conductive wires. A wireless approach may be used to overcome the problems and challenges introduced by tethered devices.

Electromagnetic (EM) and ultrasound are the two major approaches available for implantable wireless communication and energy harvesting. A challenge encountered when using EM waves to communicate with implantable devices is that the size of the associated antenna must be comparable to the wavelength of the operational frequency. State-of-the-art compact antennas rely on electromagnetic wave resonance, which leads to antenna sizes that are dependent on the electromagnetic wavelength λ₀, such that antennas typically have a size greater than λ₀/10. For instance, consider a 2 mm size antenna operating at 34 GHz frequency. For this example, the antenna size is equivalent to 0.22λ, which might be practicable for brain implantation, but the EM loss in biological tissue is extremely high at such a high frequency, and so the communication link would be quite inefficient. In fact, the EM loss between an external transceiver and an implanted device disposed the surface of the brain (grey matter) at this frequency is more than 100 dB, which is very high. Operating in a lower frequency range can resolve the EM loss problem, but a lower operational frequency means a longer wavelength and a correspondingly large antenna and implantable device.

Using ultrasound for wireless communication does not involve the antenna size problem, since the speed of acoustic waves is much slower than that of EM waves, and so the acoustic wavelength is several orders of magnitude shorter than EM wavelength at relevant acoustic frequencies. The short wavelength of ultrasound-based communication makes it feasible to utilize an ultrasound antenna that is small and operates at a low frequency. The challenge, however, of using ultrasound for brain implantable devices is that the acoustic loss in the skull is very high (e.g., 22 dB/cm/MHz). An acoustic-based implantable device operating at 10 MHz needs to contend with a 110 dB loss in the skull alone. The ultrasound approach thus requires, in addition to external transceiver and implanted device, an electromagnetic intermediate transceiver implanted in the sub-dural region to communicate with external circuitry. Implanting multiple devices in a wide region of the brain would require multiple sub-dural EM transceivers.

Furthermore, mm-size implants, both ultrasound-based and EM-based devices, generally use micro-electrodes to stimulate and record action potential of neurons. Metal electrodes, however, have been shown to suffer from several limitations. For instance, micro-electrodes are not reliable in terms of maintaining response consistency over a long period of time. This might be due to direct contact of electrodes with brain tissue, which causes reduction and oxidation at the electrode-tissue interface.

Detecting and recording magnetic fields that are generated by neuronally induced current flow may be an alternative approach to measuring neuronal action. Magnetic fields can be measured as a point source (unlike the referential needs of electrical measurements), are not disrupted by biological tissue, and have a relatively steep gradient (decreasing with the cube of the distance from the source). These features make magnetic fields a more focal and more direct measure of neuroelectrical activity than the measurement of electrical fields. Unfortunately, prior art systems sensitive enough to measure the small fields generated by brain activity are relatively large, and require cooling to reach temperatures for superconductivity.

The use of electrical stimulation to affect neural manipulation has certain advantages with respect to other known techniques. While it may not have the specificity of optogenetic approaches, electrical stimulation is a powerful and well-known method particularly in direct stimulation for research in rodents, non-human primates and, as a clinical tool, in patients. There are, however, substantial technical problems associated with electrical stimulation. One such problem is the need for both an anode and cathode, separated by some space, as the stimulating elements. In addition, inhomogeneities in the tissue with different effective electrical resistances can make the spread of current difficult to predict and even more difficult to control. As a result, electrical stimulation generally relies upon a bulk phenomenon and is neither precise nor easily tunable. Perhaps even more important is that electrical stimulation and the equipment required to produce it can change its characteristics over time and even lead to significant tissue damage. As a result, the most difficult challenge for electrical stimulation has been creating a stimulation system that avoids redox electrochemistry and glial scar accumulation from metal on cortex, and the dangers of metal deposition in neural tissue.

### SUMMARY

The embodiments described herein are directed to an implantable system (also referred to herein as an implantable device) configured to facilitate recording and manipulating neural activity, and to implement a communications link between the implantable system and an external transceiver. The implantable system utilizes an acoustically actuated magnetoelectric (ME) antenna, the resonant frequency of which changes with minute variations in local magnetic fields (e.g., magnetic fields induced by neural activity). Neural sensing may be realized by observing the resonant frequency transmitted by the ME antenna. The ME antenna can also be induced to generate steep gradients of magnetic fields.

An example embodiment of an implantable system may operate in a frequency range of the electromagnetic (EM) spectrum in which tissue loss is low (between 10 MHz and 1 GHz), and with an ME antenna that has a two-dimensional footprint of about 6000 μm² (30 μm×200 μm) and a thickness of less than 500 nm, although alternate embodiments may implement smaller dimensions. An example embodiment of an implantable system does not require contact-electrodes for recording action potential, because sensing and stimulation is implemented through magnetic fields. Rather, the example embodiment is entirely encapsulated with a biocompatible material, making it superior for long-time recording. The described embodiments of an implantable system may be sub-mm in size, and suitable for large-scale (e.g., 1,000 to 10,000 devices) neural magnetic field transmissions with high spatial and temporal resolution.

Neural stimulation using magnetic fields has substantial advantages over stimulation using electric fields. Electric field stimulation is a consequence of the local gradient of the electric potential, which is complicated by tissue conductivity. Magnetic activation, on the other hand, is sensitive to non-local field sources, but the drop in magnetic field strength occurs as the cube of the distance. This provides better spatial specificity for signal propagation, producing an effective increase in signal-to-noise ratio (SNR) at the stimulation site. Perhaps more importantly, the magnetic field is not impacted by changes in tissue composition. This makes stimulating with magnetic fields much simpler and more straightforward, and facilitates full hermetic encapsulation of the device.

Further, neural magnetic sensing is not referential, which enables significantly more compact implantable devices. Neural magnetic sensing facilitates sensing individual neuronal activity, which allows for better separation of individual neuronal activity and detection of more neurons. The implantable systems may be coated with bio-compatible polymer films such as parylene, and the same technology can be used for animals and human, allowing for direct comparisons and easier translation of animal to human information. The compact size of the implantable system allows for distributed, addressable, high channel count use in the parenchyma, pial surface, extra dural, and in both central and peripheral nervous tissue.

Neural magnetic stimulation enjoys many of the same advantages as neural magnetic sensing (e.g., being non-referential, no issue with metal deposition from contacts and no issue with heating or biofouling). In addition, neural magnet stimulation produces little or no artifact, and the ability to focus the sensing field is much easier as compared to electrical stimulation. The same implantable system may be used from in vitro and in vivo, even in humans, such that mechanisms and parameters of stimulation are easier to understand and compare.

In one aspect, an embodiment of the invention may be an implantable system comprising a magnetoelectric (ME) antenna system that is characterized by at least one resonance frequency that changes according to an ambient magnetic field strength. The implantable system may further comprise a radio frequency (RF) rectifier configured to convert RF energy, received by the ME antenna from an external source, into a direct current (DC) voltage, and to direct the DC voltage to a storage capacitor configured to provide power to a component of the implantable system. The implantable system may further comprise a transmitter configured to convey a transmission signal, through the ME antenna, to an external receiver.

The transmitter may be powered by energy stored in the storage capacitor. The ME antenna may comprise a first ME antenna element characterized by a first resonance frequency, and a second ME antenna element characterized by a second resonance frequency. The RF rectifier may be configured to convert RF energy received by the first ME antenna element, and the transmitter may be configured to convey the transmission signal through the second ME antenna.

The implantable system may further comprise a timing module that determines a time slot based on an input signal received by the ME antenna. The timing module may further comprise an oscillator and a counter. The oscillator may produce a cyclic signal and the counter may count a predetermined number of cycles of the cyclic signal to generate a time slot signal that designates a beginning of the time slot. The ME antenna, the RF rectifier, the transmitter, and other components of the implantable system may be hermetically sealed within a biocompatible material.

The RF rectifier may comprise an N-stage Dickson multiplier, wherein N is selected so that the DC voltage is compatible with an operational voltage required by the transmitter. The ME antenna may be a heterostructure that comprises a thin-film piezoelectric element and a thin-film magnetorestrictive element. The thin-film piezoelectric element may comprise AlN, and the magnetrestrictive element may comprise FeGaB. The resonance frequency may be within a range of 25 MHz to 29 MHz, or within a range of 38 MHz to 42 MHz. The ME antenna may be an ME antenna array that comprises a plurality of series-connected resonant heterostructures, each of which comprises a thin-film piezoelectric element and a thin-film magnetorestrictive element.

In another aspect, an embodiment of the invention may be a monitoring system comprising an implantable device comprising a magnetoelectric (ME) antenna, and a transceiver subsystem comprising a transmitter, a receiver, and an antenna. The transceiver subsystem may be configured to support communication with the implantable device. The monitoring system may further comprise at least one additional implantable device. The transceiver may receive sensed magnetic field information from each of (i) the implantable device and (ii) the at least one additional device. The sensed magnetic field information may comprise a first state when the transceiver detects a transmission at a resonance frequency of the ME antenna, and a second state when the transceiver does not detect the transmission at the resonance frequency of the ME antenna. Each of (i) the implantable device and (ii) the at least one additional device may be assigned a time slot for transmitting, and the transceiver may monitor the time slot for a transmission from the respective implantable device or additional device.

The transceiver subsystem may be configured to illuminate the implantable device with a magnetic field for an energy harvesting duration that is greater than one second and less than 20 seconds. The implantable device may further comprise a radio frequency (RF) rectifier configured to convert RF energy, received by the ME antenna, into a direct current (DC) voltage, and to direct the DC voltage to a storage capacitor. The implantable device may further comprise a transmitter configured to apply a transmission signal to the ME antenna.

The ME antenna may comprise a heterostructure with a thin-film piezoelectric element and a thin-film magnetorestrictive element. The thin-film piezoelectric element may comprise AlN, and the magnetrestrictive element may comprise FeGaB.

The ME antenna may be an ME antenna array comprising a plurality of series-connected resonant heterostructures, each of which comprises a thin-film piezoelectric element and a thin-film magnetorestrictive element.

In another aspect, an embodiment of the invention may comprise a method of detecting neuronal activity comprising disposing an implantable system substantially adjacent to a neuron. The implantable device may comprise (i) a magnetoelectric (ME) antenna having a resonance frequency and (ii) a transmitter. The method may further comprise monitoring the implantable system with a receiver tuned to the resonance frequency, determining that neuronal activity is absent when the receiver detects a signal from the implantable system, and determining that neuronal activity is present when the receiver does not detect the signal from the implantable system.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B illustrate conceptual block diagram and architecture of an example embodiment of the implantable system, according to the invention.

FIG. 2A depicts a simulation path loss model of a two-port communication system.

FIG. 2B depicts the simulated model depicted in FIG. 2A, between the transmit and receive antennas, as a function of frequency.

FIG. 3 shows a simulated path loss along brain layers at different frequencies.

FIG. 4A illustrates optical microscope and SEM images of an example embodiment of a ME antenna according to an embodiment of the invention.

FIG. 4B illustrates the measured electrical admittance curve associated with the example ME heterostructure shown in FIG. 4A.

FIG. 4C shows simulation results of a model corresponding to the ME heterostructure shown in FIG. 4A.

FIG. 4D shows the induced voltage of the ME heterostructure resonator shown in FIG. 4A as a function of RF magnetic field amplitude at two different excitation frequencies.

FIG. 4E shows the measured induced ME voltage output of the ME heterostructure resonator shown in FIG. 4A.

FIG. 4F shows the simulated induced ME voltage output of a simulated ME heterostructure resonator corresponding to the resonator shown in FIG. 4A.

FIGS. 5A and 5B show the DC bias field dependence of the resonance frequency of ME antenna heterostructure resonator shown in FIG. 4A.

FIG. 5C shows the Allen deviation, with respect to time, of the ME antenna heterostructure resonator shown in FIG. 4A.

FIG. 5D shows simulation results for the generated E-field gradient by a ME antenna according to the described embodiments of the invention.

FIG. 6A illustrates an example rectifier circuit according to the described embodiments of the invention.

FIG. 6B illustrates a simulated output of the example rectifier circuit of FIG. 6A.

FIG. 7 illustrates an alternative embodiment of the RF transmitter according to the described embodiments of the invention.

FIG. 8A shows an example ME antenna array with three ME antenna resonator elements.

FIG. 8B shows simulated strain energy distribution for a three-antenna array.

FIG. 8C shows the output voltage of the three-resonator-element array of FIG. 8B.

FIG. 9A shows an architecture for accomplishing external wireless power transfer and time-shared neural recording, associated with an implantable system described herein.

FIG. 9B illustrates a communication scheme between implantable systems and an external transceiver, according to embodiments of the invention.

FIG. 10 is a diagram of an example internal structure of a processing system that may be used to implement one or more of the embodiments herein.

### DETAILED DESCRIPTION

A description of example embodiments follows.

The teachings of all patents, published applications and references cited herein are incorporated by reference in their entirety.

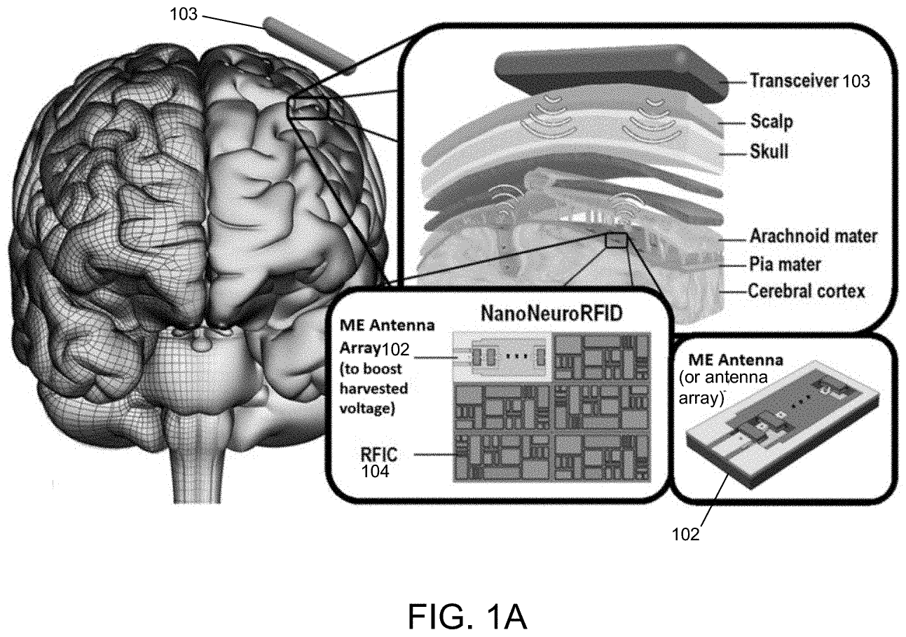

Embodiments of the implantable system described herein are directed to a wireless, sub-millimeter (mm) sized, and self-powered implantable device, which may be suitable for large-scale in-vivo neural magnetic field recording. FIGS. 1A and 1B illustrate conceptual block diagram and architecture of an example embodiment of the implantable system 100.

At the core of described implantable system 100 is an ultra-compact, acoustically actuated magnetoelectric (ME) antenna 102. The ME antenna 102, which is a nanoplate resonator (NPR), is one to two orders of magnitude smaller than comparable loop antennas, and have at least a 50 dB higher antenna gain. The ME antenna 102 is sensitive to a neural magnetic signal, in that the resonance frequency of ME antenna shifts when it is in presence of a DC or quasi-static neural magnetic field. The ME antenna 102 may be used to harvest ambient electromagnetic (EM) energy, by inducing a voltage from EM fields generated by an external transceiver 103.

The ME antenna 102 may also be used for bi-directional data communication with the external transceiver 103 (i.e., external to the immediate environment of the ME antenna). During the receiving process, the magnetic layer of the ME antenna senses H-components of EM waves, which induces an oscillating strain and a corresponding piezoelectric voltage output at the electromechanical resonance frequency. Conversely, during the transmitting process, the ME antenna produces an oscillating mechanical strain under an alternative voltage input, which mechanically excites the magnetic layer and induces a magnetization oscillation, or magnetic current, that radiates EM waves. The EM antenna therefore operates at its acoustic resonance instead of EM resonance. The acoustic wavelength is approximately five orders of magnitude shorter than the EM wavelength at the same frequency. Additional information related to the ME antenna may be found, for example, in Acoustically actuated ultra-compact NEMS magnetoelectric antennas, by, Tianxiang Nan et al., Nature communications 8, no. 1 (2017): 296.

In addition to the ME antenna 102, the implantable system 100 may comprise a lightweight and ultra-low power (ULP) integrated circuit (IC) 104 to rectify harvested RF energy and to sense a change of the resonance frequency of the ME antenna 102 for neural magnetic sensing. The ULP IC 104 may transmit data out of the brain while operating the ME antenna 102 at its nominal resonance frequency. Example embodiments of the ULP IC 104 are configured to have a power consumption low enough to be able to operate from the harvested RF energy, which eliminates the need for a battery, other than a storage capacitor to store the harvested energy.

An analysis of the tissue loss between an implantable system 100 and external transceiver begins with the general definition of path loss (PL) between a transmitter and a receiver, which is the ratio of the transmitted power to the received power:

PL|dB=10 Log(P_{T}G_{T}G_{B}/P_{R}L_{T}L_{R}), (1)

where G_{T}, L_{T}, and P_{T} are transmitter gain, feeder loss, and transmitted power, respectively, and G_{R}, L_{R}, and P_{R} are receiver gain, feeder loss, and received power, respectively. The feeder loss may be assumed to be insignificant (and thus zero) at both the transmitter and receiver. Furthermore, both antennas are the same type and have equal gain. The PL formula can therefore be simplified as follows:

$$\begin{matrix}PL \vert dB = {P}_{T} \vert dB - {P}_{R} \vert dB + {G}_{T} \vert dB i + {G}_{R} \vert dB i \\ = - \vert {S}_{21} \vert dB + 2 \times {G}_{R} \vert dB i ,\end{matrix} \qquad ( 2 )$$

where |S₂₁| is the forward transmission coefficient from port 1 to port 2, where the transmit antenna to receive antenna path is viewed as a two-port system.

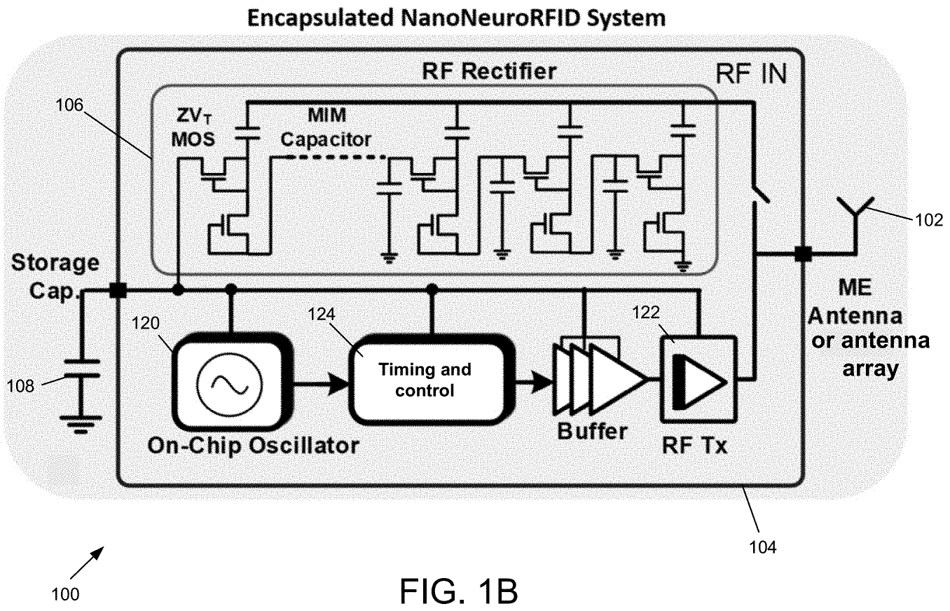

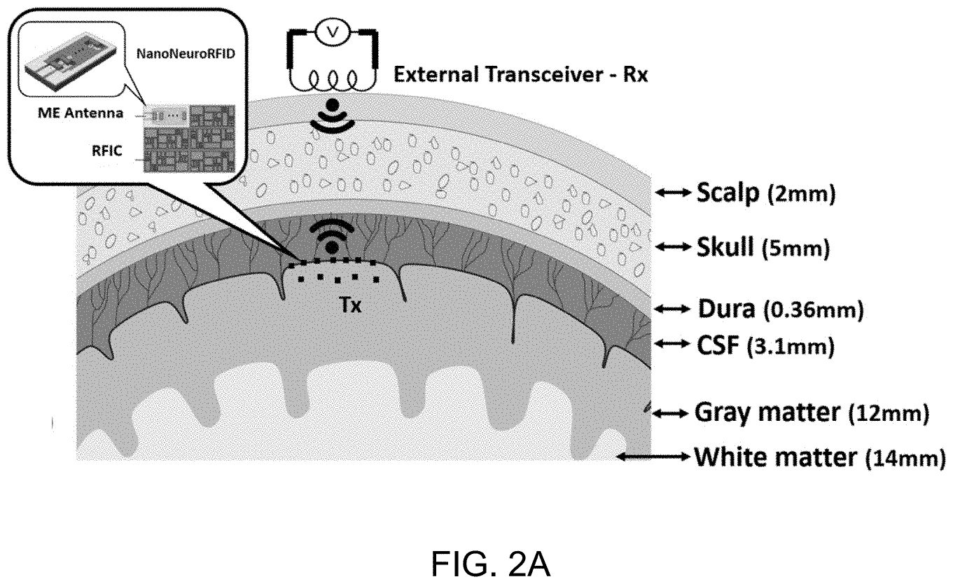

The human head has six major layers: (i) scalp, (ii) skull, (iii) dura, (iv) cerebrospinal fluid (CSF), (v) gray matter (GM), (vi) white matter, and (vii) edema. The scalp is the most external layer, and the edema is the most internal layer. We assume the receiver antenna (Rx) is outside the body and placed on the scalp surface, and that the transmitter antenna (Tx) is implanted close to GM-CSF interface. FIG. 2A depicts the simulation model of this two-port communication system. Since the Tx antenna is placed close to the GM-CSF interface, a simulation model may include all layers except the edema, because the edema is very far from the Tx port and would not have significant effect on the S₂₁ results. The simulation uses a half-wave dipole antenna for the transmit and receive antennas. In the simulation, the half-wave antenna size is half of the wavelength at every step of the simulated frequency range. The gain of this antenna set to be 2.15 dBi, so equation (2) may be rewritten as:

PL|dB=−|S₂₁|dB+4.3 dBi (3)

FIG. 2B depicts the simulated PL between the transmit and receive antennas as a function of frequency. Tissue absorption is directly proportional to conductivity of the tissue and inversely proportional to its relative permittivity. Therefore, as shown in FIG. 2B, the PL increases with higher operational frequency. The difference in dissipated power between the implantable system 100 operating at 60 MHz, and a 60 GHz conventional antenna (mm size), is more than 140 dB. A low PL is crucial for passive implantable devices since there is small amount of power available inside the body. Efficiency is even more critical when implanting a device into the brain, because the implanted component must be very small so using a battery is not acceptable due to its size and rechargeability requirements.

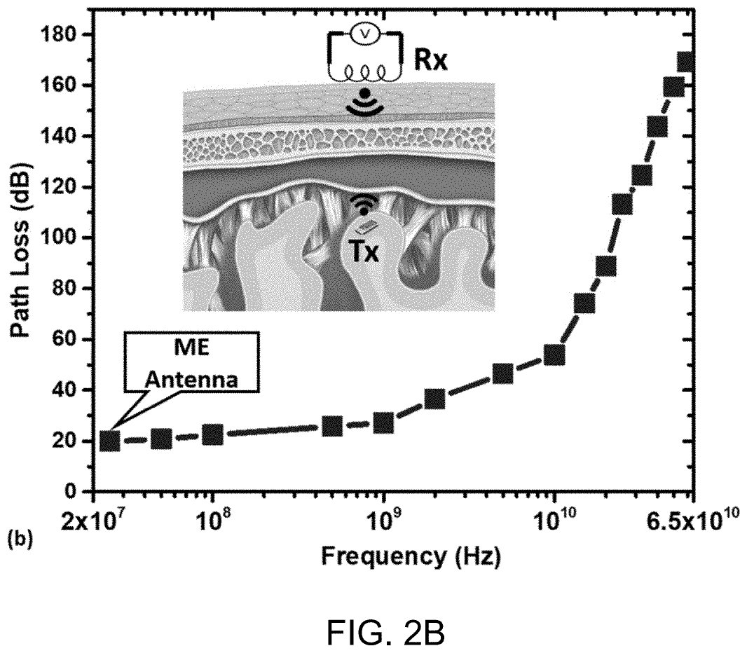

FIG. 3 shows the simulated PL along brain layers at different frequencies. Most of the loss is occurs in the scalp and CSF layers due to their high conductivity to permittivity ratio. It can be observed that the PL decreases with decreasing frequency. It is noteworthy that, even in the GHz regime, the electromagnetic loss is very small in the skull. This is a significant advantage of EM waves compared to ultrasound waves, in which the loss rate in the skull is 22 dB/cm/MHz. This implies that an acoustic-based implantable device operating at 10 MHz would have 110 dB loss in the skull alone.

Wireless implantable devices operating in the low frequency regime are therefore far more efficient than devices operating in the high frequency range because the former ones will face much less tissue loss compared to latter ones. Furthermore, a low tissue loss is especially important for passive implantable devices since there is little power available inside the body—and it is even more critical when implanting a device into the brain since the implanted component must be extremely small and using a battery is not acceptable. Implantable systems based on ME antennas as described herein provide efficiency advantages with respect to prior art implantable systems.

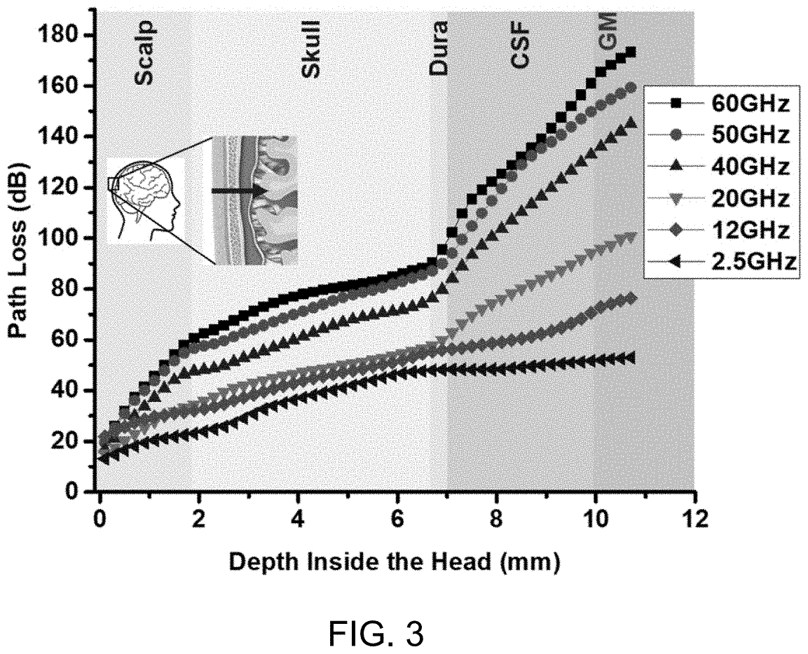

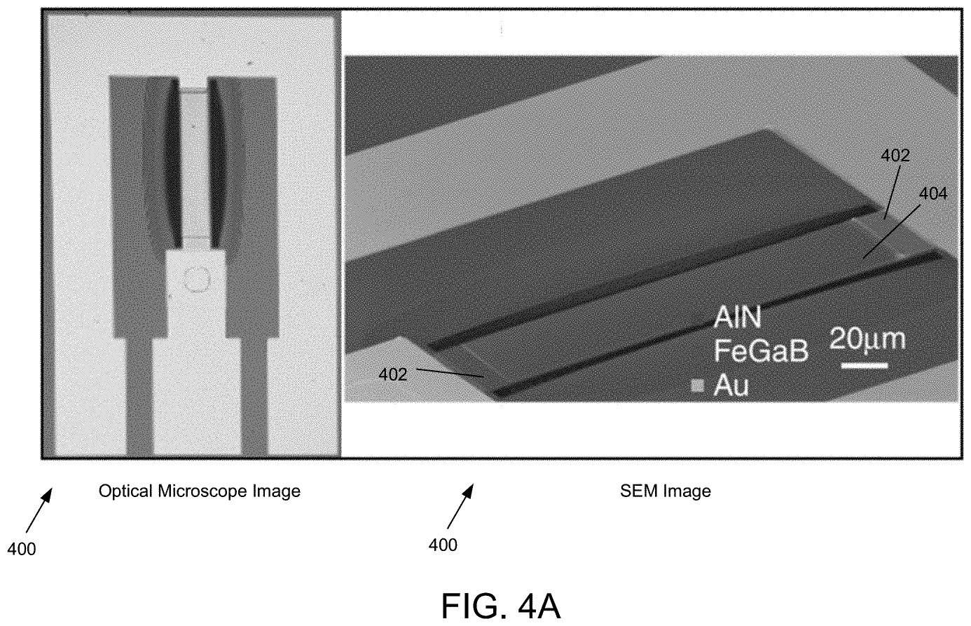

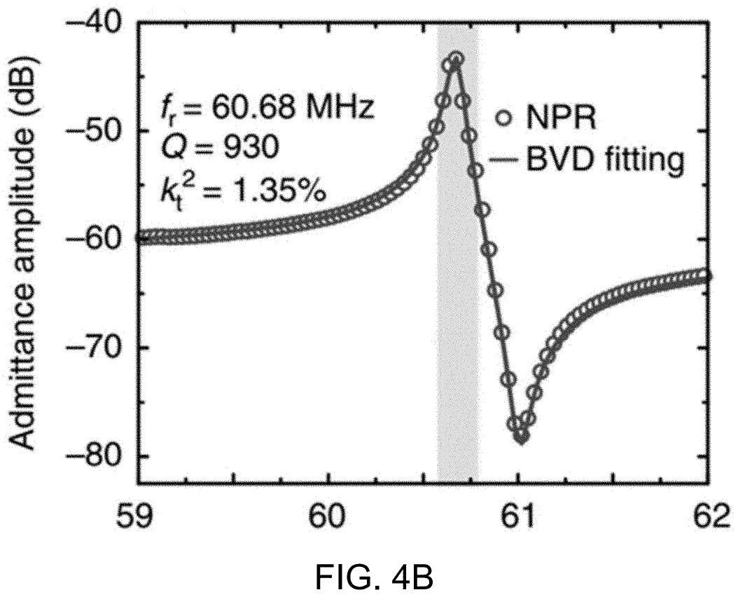

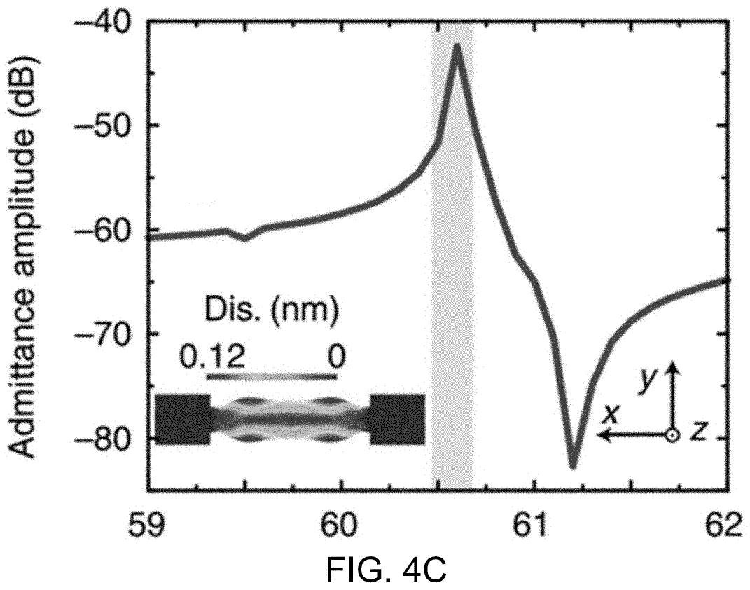

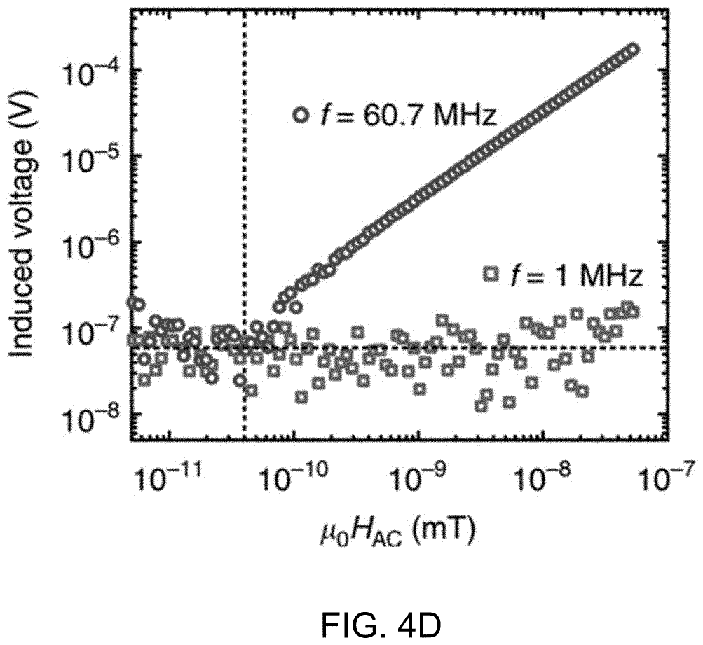

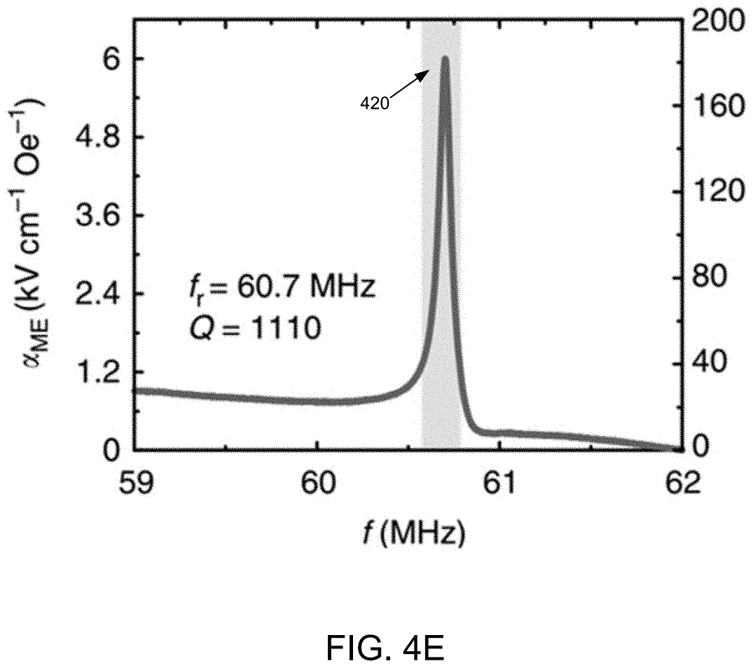

FIG. 4A illustrates optical microscope and SEM images of an example embodiment of a ME antenna 102 according to an embodiment of the invention. The ME antenna 102 may be an ME thin-film heterostructure 400, comprising thin-film piezoelectric elements 402 (e.g., 500 nm thick aluminum nitride (AlN)), and a thin-film magnetorestrictive element 404 (500 nm thick FeGaB). The ME heterostructure is suspended on a silicon substrate, as depicted in FIG. 4A. The example ME heterostructure 400 is configured for operation at 60 MHz, and has a footprint of approximately 30 μm×200 μm.

It has been shown that for magnetic induction links to implants, in a clinical environment, certain energy transfer efficiencies may exist at 27 MHz and 40 MHz. Accordingly, the described embodiments may utilize ME antennas configured with a resonant frequency within a range of 25 MHz to 29 MHz. Alternatively, the described embodiments may utilize ME antennas configured with a resonant frequency within a range of 38 MHz to 42 MHz. Alternative embodiments, however, may have other values of operative resonance frequencies.

FIG. 4B illustrates the electrical admittance curve associated with the example ME heterostructure 400 shown in FIG. 4A. The measured data of FIG. 4B matches well with simulation results in 4C. The admittance spectrum at resonance can be fitted to the Butterworth-van Dyke (BVD) model, which yields an electromechanical resonance frequency (f_{0,NPR}) of 60.68 MHz, a quality factor Q of 930, and electromechanical coupling coefficient (kₜ²) of 1.35%. Together, these parameters indicate a high electromechanical transduction efficiency and low loss. This electromechanical resonance frequency corresponds to the contour mode of vibration excited in AlN, which can be analytically expressed as f_{0,NPR}∝1/(2W₀\sqrt{E/ρ}), where W₀ is the width of the resonator pitch, E and ρ are the equivalent Young's modulus and equivalent density, respectively, of the FeGaB and AlN heterostructure resonator. The quality factor Q of this ME resonator is much higher than for conventional low frequency ME heterostructures.

Under the excitation of an RF magnetic field with an amplitude of H_{rf}˜60 nT (provided, for example, by an external RF coil) along the length direction of the resonator, the induced ME voltage output of the ME heterostructure resonator 400 (measured with an ultra-high frequency lock-in amplifier) is shown in FIG. 4E. A clear electromechanical resonance peak 420 can be observed in the ME voltage spectrum at about 60.7 MHz with a peak amplitude of 180 μV. The induced output voltage from the ME antenna can be directly used for energy harvesting. The amplitude of the peak is very sensitive to the excitation frequency, exhibiting a Q similar to the admittance curve in FIG. 4B. The experimentally measured output ME voltage spectrum, shown in FIG. 4E, agrees well with simulation results, shown in FIG. 4F. A high ME coupling coefficient of α=∂U/(∂H_{rf}d)=6 kV Oe⁻¹cm⁻¹ (see, e.g., FIG. 4E) can be derived at the electromechanical resonance frequency, where U is the induced voltage ME voltage, H_{rf} is the applied alternative magnetic field, and d is the thickness of AlN layer. Note that a high a is obtained at zero DC magnetic field, and is comparable to recent reported α values with optimum bias magnetic field. FIG. 4D shows the induced voltage as a function of RF magnetic field amplitude at two different excitation frequencies. At the resonance frequency of 60.7 MHz (shown with circles), the example ME antenna shows a detection limit of 40 pT for the NPR ME sensor in unshielded lab environment.

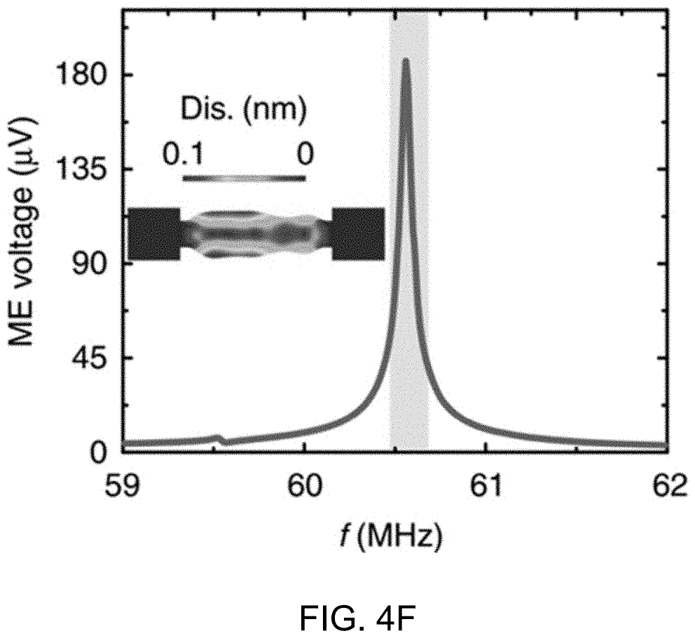

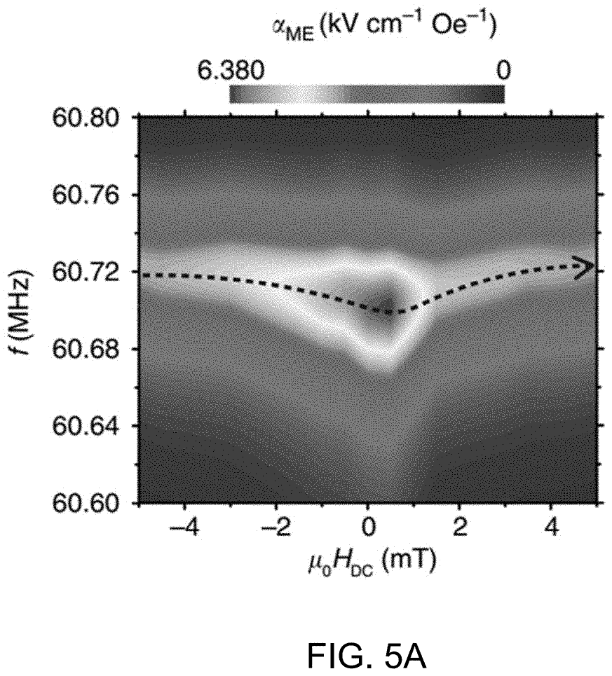

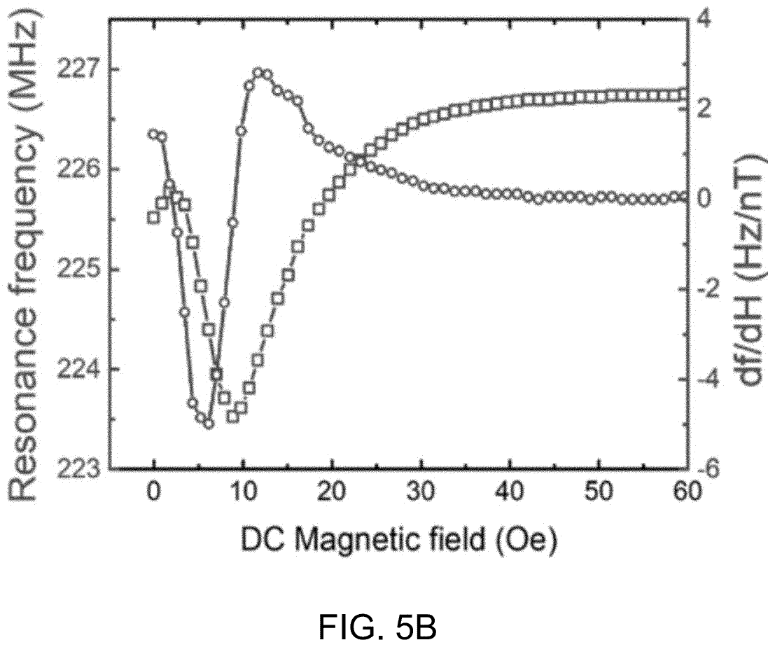

Neuronal activity produces small transient currents that produce small neuronal magnetic fields (NMFs). A NMF can be detected by an ME antenna because the electromechanical resonance frequency (or operational frequency) of the ME antenna is a substantial function of even a weak DC bias magnetic field, as shown in FIGS. 5A and 5B. This characteristic can be attributed to the delta-E (ΔE) effect, in which the bias magnetic field modifies the Young's modulus of FeGaB, which leads to a modified electromechanical resonance frequency of the FeGaB and AlN heterostructure resonator. The DC bias field dependence of the resonance frequency of ME antenna heterostructure resonator, shown in FIGS. 5A and 5B, enables neural magnetic sensing through shifted resonance frequency of magnetoelectric antennas in the presence of neural magnetic fields. These RF magnetoelectric NPRs based on magnetoelectric FeGaB/AlN thin-film heterostructure exhibit an ultra-low magnetic noise of 1.58 pT/Hz^{1/2} at 10 Hz, and a sensitivity of 4.98 Hz/nT, as shown in FIGS. 5A and 5B. They also have a low Allan deviation of 0.0245 Hz at 10 Hz, and a low noise level of 1.58 pT/Hz^{1/2} at 10 Hz, as can be seen in FIG. 5C. The limit of detection of these magnetoelectric sensors has been demonstrated to be 200 pT for quasi-static magnetic fields, which is about 1 to 2 orders of magnitude better than the well-known nanoscale magnetometers such as GMR (giant magnetoresistive) or TMR (tunneling magnetoresistive) magnetometers.

The capability of the described magnetoelectric (ME) antennas to receive and transmit electromagnetic waves at their acoustic resonance frequencies is referred to herein as the ME effect. The acoustic resonance in ME antennas stimulates magnetization dynamics in the ferromagnetic thin film, which results in the radiation of electromagnetic waves. Vice versa, ME antennas sense the magnetic fields of electromagnetic waves, producing a piezoelectric voltage output, which can be used for wireless energy harvesting. The ME antennas with sizes of λ₀/1000 to λ₀/100 are achievable, which demonstrates 1-2 orders of magnitude size miniaturization over prior art compact antennas, and 50 dB higher gain as compared to that of the same-size small loop antenna. The described ME antennas may be used for ultra-compact bio-implantable sensing and stimulation systems, and for Internet of Things (IoT) applications, among others.

A wide variety of different theoretical and empirical approaches to understanding the magnetic fields generated from neurons have been undertaken. For example, a superconducting quantum interference device (SQUID) magnetometer has been used to measure the magnetic field generated by action potentials in an isolated frog sciatic nerve as 1200 pT at 1.3 mm from the nerve. These and other studies have shown a wide range of different neuronal magnetic field (NMF) estimates dependent on the parameters of the model, type of activity modeled (e.g., subthreshold synaptic input vs. suprathreshold action potentials), and model assumptions, with estimates varying as much as 1000-fold. Other estimates can be deduced from measurements being made with EEG (electroencephalography) and MEG (magnetoencephalography). Approximately 50,000 active neurons are needed to generate a signal that is detectable by SQUID magnetometers, which are ˜10 fT for cortical evoked activity and ˜103 fT for the human alpha rhythm at a distance of about 50 mm from neuron currents (e.g., outside the skull where MEG sensors are placed. Since the fringing magnetic field H generated from the neuron currents is inversely proportional to the third power of the distance from the dipole r, i.e., H∝1/r³, the magnetic fields from neuronal activity are significantly increased from 0.1˜10 pT measured outside the skull (about 20 mm from the neuron current dipoles) to 100˜10000 pT at a distance of 2 mm from the generators (e.g., at the pial surface, for example). The magnetoelectric NPR-based magnetoelectric antenna of the described embodiments, sensitive to at least 200 pT, is adequate to detect neuronal magnetic signals at the pial surface where they are 1-2 mm from the field generators for layers II, III cell bodies and all of the superficial dendrites of neurons. They will have even higher signal-to-noise capabilities in the parenchyma where they will be in contact with, or within hundreds of μm of many neuronal soma.

An important parameter relevant to neuron stimulation capability of a device is gradient of induced electric field (E-field). The threshold of E-field gradient is about 11 kV/m² so that the stimulator device should be able to generate more than this 11 k V/m² to be able to fire the neurons. FIG. 5D shows the simulation results for the generated E-field gradient by a ME antenna according to the described embodiments. As shown, the gradient of electric field gradient (dE_{y}/dy) in the four corner regions 502, 504, 506, 508, of the magnetoelectric antenna is as high as 1×10⁶ V/m² at the four corners of the ME antenna, which is much higher than the threshold value 11 kV/m², and about 1 to 2 orders of magnitude higher than what can be achieved with a micro-coil on a neural magnetic probe for neural magnetic stimulation. The large spatial regions with electric field gradients above the threshold value are each greater than or equal to 150 μm×150 μm, which is sufficient to facilitate neural magnetic stimulation.

Referring again to FIG. 1B, an on-chip RF rectifier circuit 106 of the implantable system 100 may be used to harvest energy from an RF signal received through ME antenna array 102. The rectifier circuit 106 may generate a DC output voltage based on the incoming power from the RF source. The DC voltage is stored on a capacitor 108, which will act as a buffer to supply energy to the on-chip circuits, including during RF transmission. The RF rectifier 106 multiplies the low voltage (e.g., 2-3 mV received using the ME antenna array) AC signal to generate a high voltage DC signal. Once a sufficient voltage has developed on the storage capacitor, the on-chip RF transmitter using the ME antenna can transmit the signal to the external transceiver. The resonance frequency of ME antenna depends on local neuronal activity. The antenna matching network is designed such that the absence of neural activity results in a transmission at a nominal frequency of an example ME antenna, e.g., 27.12 MHz, which is interpreted as “off” (i.e., no neural activity detected). The presence of neural activity, on the other hand, shifts the resonance frequency of ME antenna, so the transmitted power is small and the receiver interprets the signal as “on” (i.e., neural activity detected). Hence, the external data acquisition device can decode the neuron activity with an on-off keying (OOK) scheme.

The rectifier circuit 106 receives an RF signal from the ME antenna array 102 and converts the RF signal into a DC level. The rectifier circuit 106 also provides a gain for the received signal to become a sufficient DC voltage for the use of on-chip circuits.

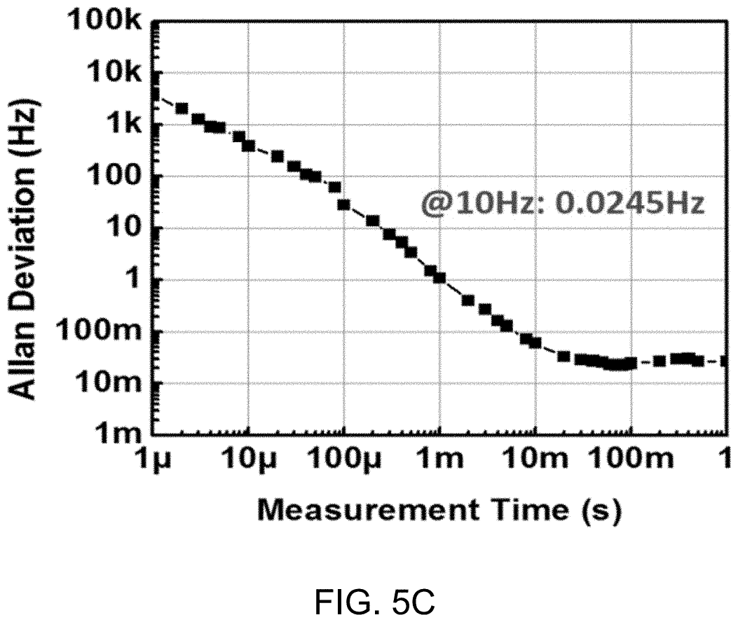

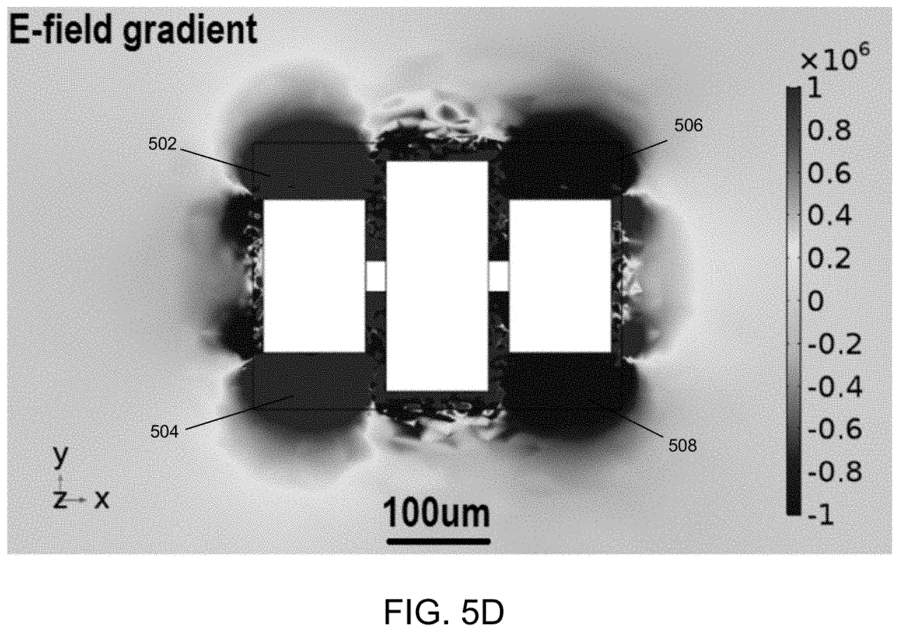

An example rectifier circuit 106, shown in FIG. 6A, depicts a portion of a 20-stage Dickson multiplier. The example rectifier circuit 106 comprises a series-connected set of twenty rectifier stages, only three of which (602, 604, 606) are explicitly shown. Alternative embodiments of the rectifier circuit 106 may utilize other quantities of rectifier stages.

To improve the performance of the rectifier, the flying capacitors used at the outputs of the rectifier stages are metal-insulator-metal (MIM) capacitors, as they have very low parasitic component when compared to other capacitors that can be integrated.

The example rectifier circuit 106 operates by passing the positive portion of the received RF signal through the first rectifier stage 602, which charges the output first stage 602 to the amplitude level of the received signal. For the next stage 604, the received RF signal will swing on top of the DC output level of the first stage 602, which charges the output of the second stage 604 output to twice the amplitude of the received RF signal. Each succeeding stage amplifies the amplitude of the received RF signal to a higher DC value. The example rectifier circuit 106 shown in FIG. 6A uses 180 nm complementary metal-oxide-semiconductor (CMOS) technology, although other fabrication technologies may alternatively be used. The example rectifier circuit 106 utilizes low-threshold voltage (LV_{T}) transistors that act as diodes, each of which has very low cut-in voltage operating in the sub-threshold region of the LV_{T} transistor. FIG. 6B illustrates the simulated output 608 of the example rectifier circuit 106 with an incoming RF signal (RF IN) with an amplitude 2.5 mV. As FIG. 6B shows, the output 608 reaches 0.7V just after 1 ms, and reaches 1.2V at about 9 ms. In an example embodiment, the storage capacitor 108 is a 2 nF capacitor, although other value of the storage capacitor 108 may alternatively be used. The specific value of the storage capacitor 108 depends on system requirements, e.g., a larger capacitor allows the transmitter 122 and other components of the system 100 to run longer for each energy harvest period, but the size of the implantable system 100 will necessarily limit the size of the storage capacitor 108.

The implantable system 100 may further comprise a precision clock source configured to provide timing information on the IC so that the implantable system 100 may be properly synchronized. Integrated circuits commonly utilize off-chip crystal oscillators for providing such timing information, but such an oscillator is impractical for implantable devices. The described embodiments of an implantable system 100 use a highly stable, on-chip relaxation oscillator (RO) 120, in which a proportional-to-absolute temperature (PTAT) current reference and a complementary current source are combined to offset any temperature dependence. The resulting temperature-compensated current source is used for the on-chip, current-source-based oscillator 120, and has a very low temperature variation (e.g., less than a 5 ppm/° C. temperature coefficient).

The implantable system 100 may further comprise an RF Transmitter 122. Once enough power has been stored on the storage capacitor 108, the implantable system 100 may sense neuron activity through the ME antenna. The resonance frequency of the antenna will move due to the underlined neural activity. The RF transmitter 122 transmits a signal using the ME antenna 102, the carrier frequency of which is dependent on ambient magnetic fields generated by neural activity. An external receiving device may acquire the transmitted signal and sense the change in the carrier frequency as indicative of neural activity.

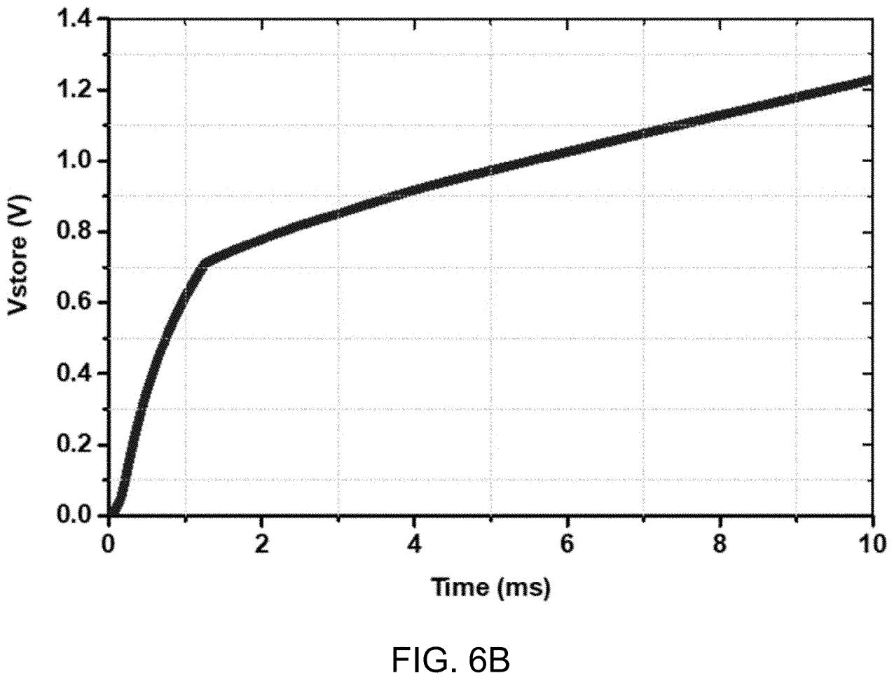

FIG. 7 illustrates an alternative embodiment of the RF transmitter 122. In this embodiment, the ME antenna 102 is employed as resonating component of the RF transmitter 122. Along with the parallel on-chip capacitors C₁ and C₂, the ME antenna 102 forms a Pierce oscillator architecture. The start-up time of the RF transmitter 122 will be in the hundreds of microseconds range, and the RF transmitter 122 may transmit neuronal activity for about 1 millisecond, expending a total of about 1 nJ of energy that is supplied by the storage capacitor 108. Embodiments of the RF transmitter 122 may be configured in a higher output power mode for neural stimulation. An advantage of having a predetermined amount of energy stored on the storage capacitor 108 is that neural stimulation may be accomplished with a known energy, for known duration of time, and in a very precise manner.

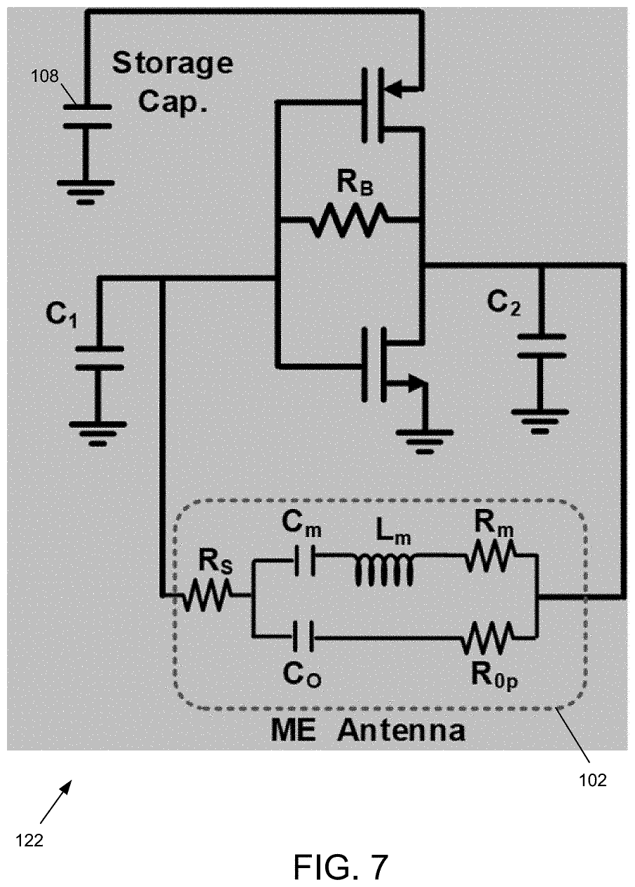

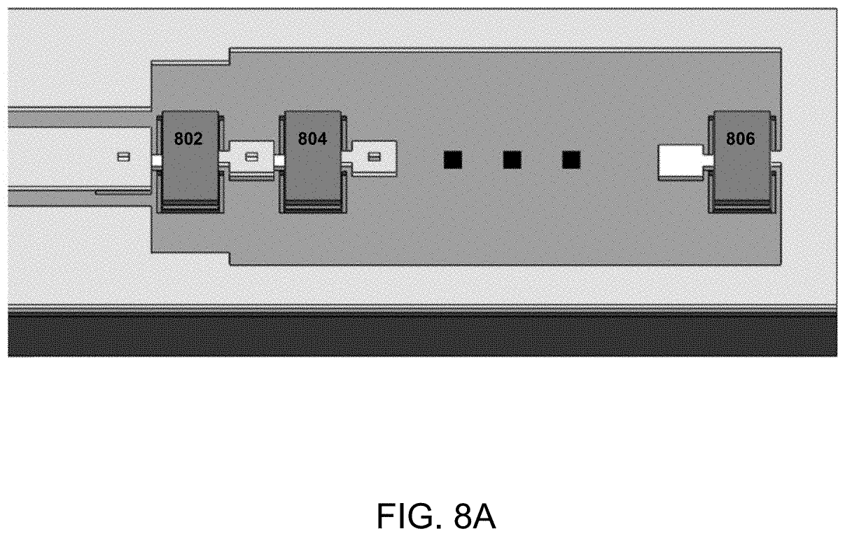

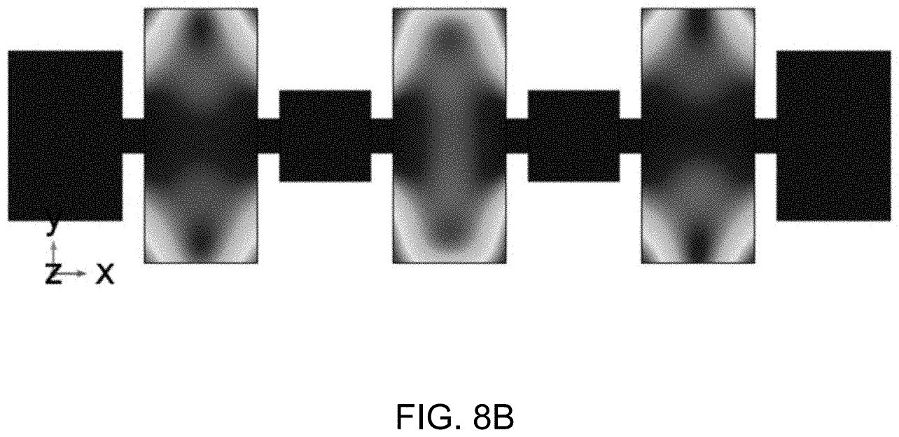

As described herein, a single ME antenna has a relatively small footprint (e.g., 30 μm×200 μm). Consequently, relatively low voltage or power can be harvested with a single ME antenna. More energy can be harvested using ME antenna arrays, which can achieve a higher output voltage under RF magnetic field excitation because ME antennas in the array all operate in-phase. In an example embodiment, each ME antenna element of the array has the size of 100 μm×200 μm, and the IC of the implantable system is about 1 mm×1 mm. Further, the ME antennas of the array can be made to overlap part of the RFICs to reduce cost. FIG. 8A shows an example ME antenna array with three ME antenna resonator elements; first resonator element 802, second resonator element 804, and third resonator element 806. FIG. 8B shows simulated strain energy distribution for a three-antenna array (with the highest strain occurring at the corners of the resonators), and FIG. 8C shows the output voltage of the three-resonator-element array of FIG. 8B, under a 200 nT magnetic field excitation (which is well within FCC limit) at 27.12 MHz. FIG. 8C shows significantly improved voltage output as compared to the array with 1 and 2 resonator elements. FIG. 8C demonstrates that a ME array with three ME resonator elements can lead to more than three times the output voltage (compared to a single ME resonator) due to non-linear coupling. An antenna array with 5 to 10 ME resonator elements may produce in the range of 2 to 3 mV output voltage, which is suitable for energy harvesting.

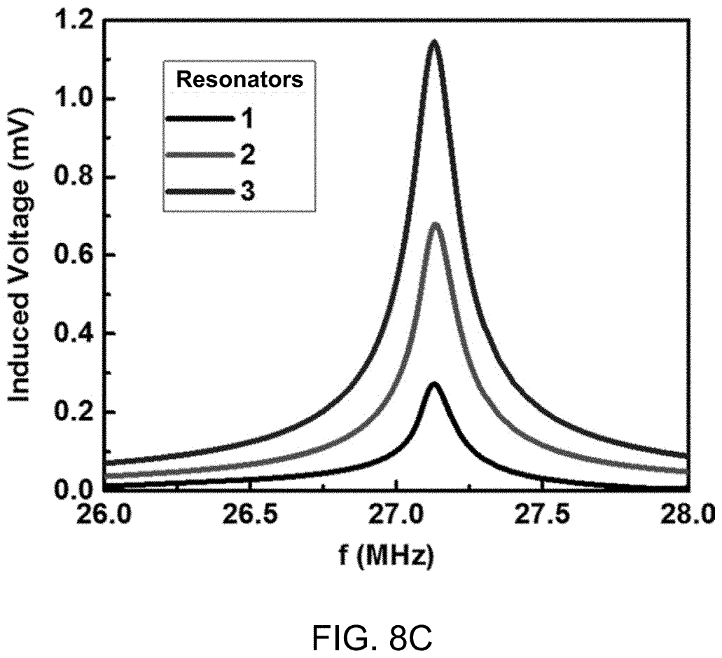

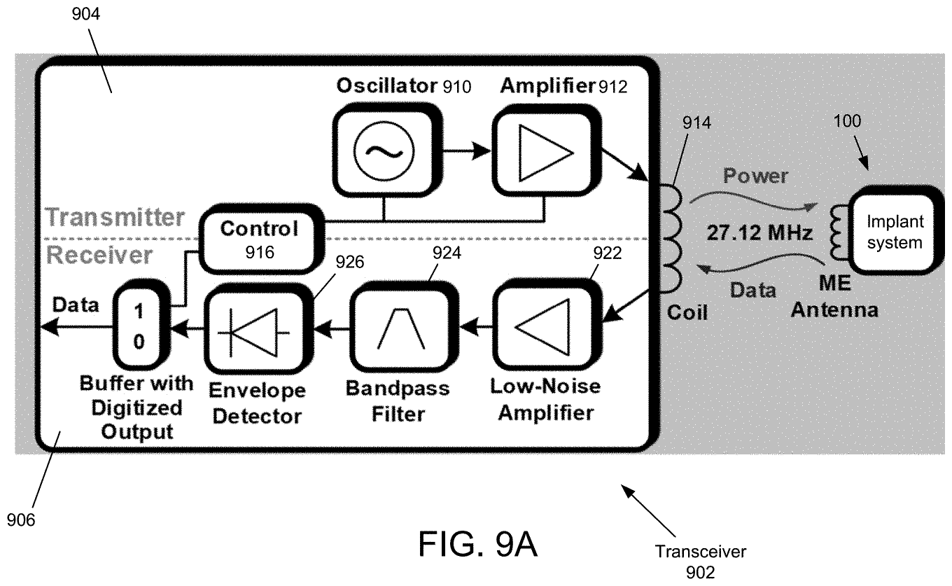

FIG. 9A shows an example architecture for accomplishing external wireless power transfer and time-shared neural recording, associated with an implantable system 100, according to embodiments of the invention. A transceiver 902 may comprise a transmitter section 904 and a receiver section 906. In the transmitter section 904, an oscillator 910 provides a 27.12 MHz signal to an amplifier 912, and the amplifier 912 amplifies the signal and drives the amplified signal to an antenna coil 914 with up to −10 dBm of output power for wireless power transfer to the implantable systems 100. As described herein, an example ME antenna may have a resonance frequency of approximately 27.12 MHz, which is matched to the frequency of the oscillator 910. As set forth herein, due to energy transfer efficiencies, there may be advantages to operating the implantable systems 100 at either 27 MHz or 40 MHz (+/−2 MHz). It should be understood, however, that the example embodiments configured to operate in either of these frequency ranges are not intended to be limiting. Other resonance frequency for the ME antennas may alternatively be used.

FIG. 9B illustrates a communication scheme in which all implantable systems 100 are simultaneously charged through wireless power transmission from the external transceiver. Afterwards, the external transceiver will sequentially receive data transmissions from implantable systems 100 for a duration of 100 ms, in which each implantable system 100 has a specified time slot (TS) of 18.44 μs. During its respective time slot, each implantable system 100 may transmit via OOK, where the “on” signal indicates the detection of neuron firing and the “off” signal indicates neuron inactivity. In this example embodiment, 5000 implantable systems 100 are shown communicating with the external transceiver 902, although that number of implantable systems is for descriptive purposes only and is not intended to be limiting. Hence, the external transceiver 902 may record data at a total rate of 50,000 bits per second with the proposed prototype system. For each TS transmission by an implant system 100, an example receiver 906 may amplify the received signal with a low noise amplifier 922, filter the amplified signal with a bandpass filter 924 that frames the resonance bandwidth of the implant system's ME antenna, and detect the presence of a transmission from the implantable system 100 using an envelope detector 926. Other embodiments may implement a receiver for detecting implantable system transmission in keeping with the concepts described herein, but using other components known in the art.

The moment 920 at which the power transmission stops marks the reference for the timing circuits on the implantable system 100 and the external transceiver 902. A code programmed into the timing circuit 124 of each implantable system 100 serves as its address in the time division multiple access (TDMA) scheme. Using the moment 920 as a reference, the timing circuit will trigger the RF transmitter 122 at its respective TS based on its programmed code. For this example communications link, the TS for each implantable system 100 is 18.44 μs (which is 500 periods of the 27.12 MHz oscillator). The code programmed into the implantable system 100 may be programmed when the implantable system 100 is fabricated, or it may be programmed by transmitting the code to the implantable system 100 through the ME antenna 102. In this latter case, receiver component in the implantable system 100 (not explicitly shown in FIG. 1B) may be selectively coupled to the ME antenna to receive the code and store the code in appropriate storage facilities in the timing and control component 124.

The external transceiver 902 determines, for each implantable system 100, an OOK state by evaluating the received signal during the time slot associated with that implantable system 100. The presence of numerous cycles of RF energy during that time slot designates the “on” state, while the absence of an RF signal designates an “off” state. With the 100 ms long recording mode as shown in FIG. 9B, up to 5424 time slots could be accommodated with this example scheme, and even more if the TS duration is reduced.

An example embodiment of the external transceiver 902 in FIG. 9A may be configured as a custom system-on-a-chip (SOC), with a footprint of less than 25 mm² and power consumption below 5 mW. This example external transceiver embodiment may be configured for disposition in a head-mountable device adapted for use in a clinical, wireless medical application.

In an application in which uninterrupted neural recording and/or manipulation are desired, an external transceiver device 902 may comprise a 40.68 MHz transmitter in addition to the 27.12 MHz transmitter for continuous power transmission to implantable systems 100. In such an embodiment, each of the implantable systems 100 includes two ME antennas, one of which is resonant at one of the transmitter frequencies (e.g., 40.68 MHz for energy harvesting), and the other of which is resonant at the other transmitter frequency (e.g., 27.12 MHz for data transfer). As mentioned elsewhere herein, the specific transmitter frequencies may be selected to take advantage of certain efficiencies inherent in the particular application, although other embodiments may operate at other frequencies within a frequency band (e.g., between 10 MHz and 1 GHz).

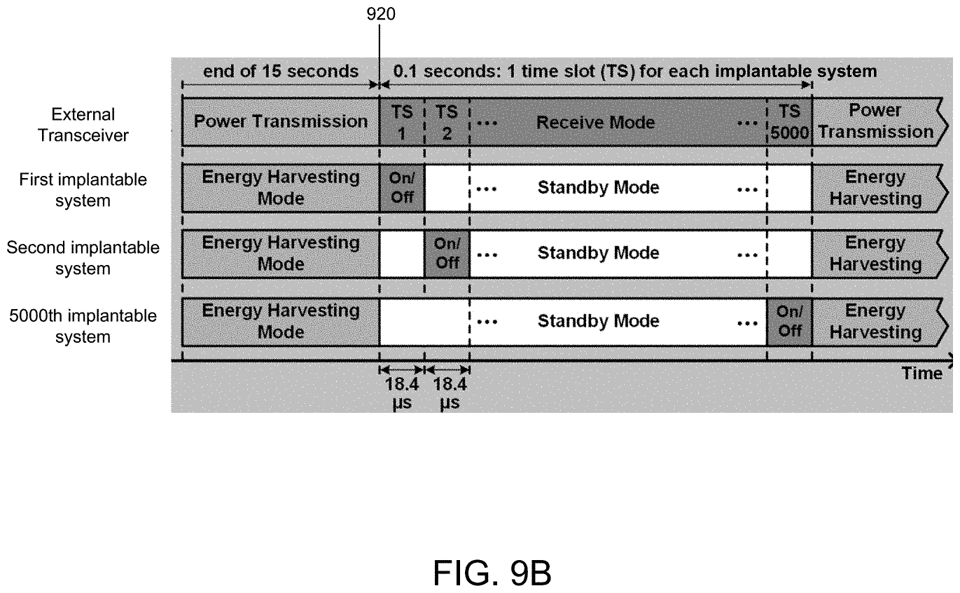

FIG. 10 is a diagram of an example internal structure of a processing system 1000 that may be used to implement one or more of the embodiments herein. Each processing system 1000 contains a system bus 1002, where a bus is a set of hardware lines used for data transfer among the components of a computer or processing system. The system bus 1002 is essentially a shared conduit that connects different components of a processing system (e.g., processor, disk storage, memory, input/output ports, network ports, etc.) that enables the transfer of information between the components.

Attached to the system bus 1002 is a user I/O device interface 1004 for connecting various input and output devices (e.g., keyboard, mouse, displays, printers, speakers, etc.) to the processing system 1000. A network interface 1006 allows the computer to connect to various other devices attached to a network 1008. Memory 1010 provides volatile and non-volatile storage for information such as computer software instructions used to implement one or more of the embodiments of the present invention described herein, for data generated internally and for data received from sources external to the processing system 1000.

A central processor unit 1012 is also attached to the system bus 1002 and provides for the execution of computer instructions stored in memory 1010. The system may also include support electronics/logic 1014, and a communications interface 1016. The communications interface 1016 may, for example, communicate with the transmitter 904 and/or the receiver 906 of the transceiver 902 as a component of the control 916, as described with reference to FIG. 9A.

In one embodiment, the information stored in memory 1010 may comprise a computer program product, such that the memory 1010 may comprise a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection.

It will be apparent that one or more embodiments described herein may be implemented in many different forms of software and hardware. Software code and/or specialized hardware used to implement embodiments described herein is not limiting of the embodiments of the invention described herein. Thus, the operation and behavior of embodiments are described without reference to specific software code and/or specialized hardware—it being understood that one would be able to design software and/or hardware to implement the embodiments based on the description herein.

Further, certain embodiments of the example embodiments described herein may be implemented as logic that performs one or more functions. This logic may be hardware-based, software-based, or a combination of hardware-based and software-based. Some or all of the logic may be stored on one or more tangible, non-transitory, computer-readable storage media and may include computer-executable instructions that may be executed by a controller or processor. The computer-executable instructions may include instructions that implement one or more embodiments of the invention. The tangible, non-transitory, computer-readable storage media may be volatile or non-volatile and may include, for example, flash memories, dynamic memories, removable disks, and non-removable disks.

While example embodiments have been particularly shown and described, it will be understood by those skilled in the art that various changes in form and details may be made therein without departing from the scope of the embodiments encompassed by the appended claims.

What is claimed is:

1. An implantable system, comprising:
a magnetoelectric (ME) antenna system characterized by at least one resonance frequency that changes according to an ambient magnetic field strength;
a radio frequency (RF) rectifier configured to convert RF energy, received by the ME antenna from an external source, into a direct current (DC) voltage, and to direct the DC voltage to a storage capacitor configured to provide power to a component of the implantable system; and
a transmitter configured to convey a transmission signal, through the ME antenna, to an external receiver.

2. The implantable system of claim 1, wherein the transmitter is powered by energy stored in the storage capacitor.

3. The implantable system of claim 1, wherein the ME antenna comprises a first ME antenna element characterized by a first resonance frequency, and a second ME antenna element characterized by a second resonance frequency, wherein the RF rectifier is configured to convert RF energy received by the first ME antenna element, and the transmitter is configured to convey the transmission signal through the second ME antenna.

4. The implantable system of claim 1, further comprising a timing module that determines a time slot based on an input signal received by the ME antenna.

5. The implantable system of claim 1, the timing module further comprising an oscillator and a counter, wherein the oscillator produces a cyclic signal and the counter counts a predetermined number of cycles of the cyclic signal to generate a time slot signal that designates a beginning of the time slot.

6. The implantable system of claim 1, wherein the ME antenna, the RF rectifier, the transmitter, and other components of the implantable system, are hermetically sealed within a biocompatible material.

7. The implantable system of claim 1, wherein the RF rectifier comprises an N-stage Dickson multiplier, wherein N is selected so that the DC voltage is compatible with an operational voltage required by the transmitter.

8. The implantable system of claim 1, wherein the ME antenna is a heterostructure that comprises a thin-film piezoelectric element and a thin-film magnetorestrictive element.

9. The implantable system of claim 8, wherein the thin-film piezoelectric element comprises AlN, and the magnetrestrictive element comprises FeGaB.

10. The implantable system of claim 1, wherein the resonance frequency is within a range of 25 MHz to 29 MHz, or within a range of 38 MHz to 42 MHz.

11. The implantable system of claim 1, wherein the ME antenna is an ME antenna array comprising a plurality of series-connected resonant heterostructures, each of which comprises a thin-film piezoelectric element and a thin-film magnetorestrictive element.

12. A monitoring system, comprising:
an implantable device comprising a magnetoelectric (ME) antenna; and
a transceiver subsystem comprising a transmitter, a receiver, and an antenna, the transceiver subsystem configured to support communication with the implantable device.

13. The monitoring system of claim 12, further comprising at least one additional implantable device, wherein the transceiver receives sensed magnetic field information from each of (i) the implantable device and (ii) the at least one additional device.

14. The monitoring system of claim 13, wherein the sensed magnetic field information comprises a first state when the transceiver detects a transmission at a resonance frequency of the ME antenna, and a second state when the transceiver does not detect the transmission at the resonance frequency of the ME antenna.

15. The monitoring system of claim 12, wherein each of (i) the implantable device and (ii) the at least one additional device is assigned a time slot for transmitting, and the transceiver monitors the time slot for a transmission from the respective implantable device or additional device.

16. The monitoring system of claim 12, wherein the transceiver subsystem is configured to illuminate the implantable device with a magnetic field for an energy harvesting duration that is greater than one second and less than 20 seconds.

17. The monitoring system of claim 12, wherein the implantable device further comprises (i) a radio frequency (RF) rectifier configured to convert RF energy, received by the ME antenna, into a direct current (DC) voltage, and to direct the DC voltage to a storage capacitor, and (ii) a transmitter configured to apply a transmission signal to the ME antenna.

18. The monitoring system of claim 12, wherein the ME antenna comprises a heterostructure with a thin-film piezoelectric element and a thin-film magnetorestrictive element.

19. The monitoring system of claim 18, wherein the thin-film piezoelectric element comprises AlN, and the magnetrestrictive element comprises FeGaB.

20. The monitoring system of claim 12, wherein the ME antenna is an ME antenna array comprising a plurality of series-connected resonant heterostructures, each of which comprises a thin-film piezoelectric element and a thin-film magnetorestrictive element.

21. A method of detecting neuronal activity, comprising:
disposing an implantable system substantially adjacent to a neuron, the implantable device comprising (i) a magnetoelectric (ME) antenna having a resonance frequency and (ii) a transmitter;
monitoring the implantable system with a receiver tuned to the resonance frequency; and
determining that neuronal activity is absent when the receiver detects a signal from the implantable system, and determining that neuronal activity is present when the receiver does not detect the signal from the implantable system.
